SEQUENCE LISTING (2 sequences)

SEQ ID NO: 1 - DNA, 2020 bp, Homo sapiens
  steroid and xenobiotic sensing nuclear receptor (SXR)
ggcacgaggagatctaggttcaaattaatgttgcccctagtggtaaaggacagagaccctcagactgatgaaatgcgctcagaattacttagacaaagcggatatttgccactctcttccccttttcctgtgtttttgtagtgaagagacctgaaagaaaaaagtagggagaacataatgagaacaaatacggtaatctcttcatttgctagttcaagtgctggacttgggacttaggaggggcaatggagccgcttagtgcctacatctgacttggactgaaatataggtgagagacaagattgtctcatatccggggaaatcataacctatgactaggacgggaagaggaagcactgcctttacttcagtgggaatctcggcctcagcctgcaagccaagtgttcacagtgagaaaagcaagagaataagctaatactcctgtcctgaacaaggcagcggctccttggtaaagctactccttgatcgatcctttgcaccggattgttcaaagtggaccccaggggagaagtcggagcaaagaacttaccaccaagcagtccaagaggcccagaagcaaacctggaggtgagacccaaagaaagctggaaccatgctgactttgtacactgtgaggacacagagtctgttcctggaaagcccagtgtcaacgcagatgaggaagtcggaggtccccaaatctgccgtgtatgtggggacaaggccactggctatcacttcaatgtcatgacatgtgaaggatgcaagggctttttcaggagggccatgaaacgcaacgcccggctgaggtgccccttccggaagggcgcctgcgagatcacccggaagacccggcgacagtgccaggcctgccgcctgcgcaagtgcctggagagcggcatgaagaaggagatgatcatgtccgacgaggccgtggaggagaggcgggccttgatcaagcggaagaaaagtgaacggacagggactcagccactgggagtgcaggggctgacagaggagcagcggatgatgatcagggagctgatggacgctcagatgaaaacctttgacactaccttctcccatttcaagaatttccggctgccaggggtgcttagcagtggctgcgagttgccagagtctctgcaggccccatcgagggaagaagctgccaagtggagccaggtccggaaagatctgtgctctttgaaggtctctctgcagctgcggggggaggatggcagtgtctggaactacaaacccccagccgacagtggcgggaaagagatcttctccctgctgccccacatggctgacatgtcaacctacatgttcaaaggcatcatcagctttgccaaagtcatctcctacttcagggacttgcccatcgaggaccagatctccctgctgaagggggccgctttcgagctgtgtcaactgagattcaacacagtgttcaacgcggagactggaacctgggagtgtggccggctgtcctactgcttggaagacactgcaggtggcttccagcaacttctactggagcccatgctgaaattccactacatgctgaagaagctgcagctgcatgaggaggagtatgtgctgatgcaggccatctccctcttctccccagaccgcccaggtgtgctgcagcaccgcgtggtggaccagctgcaggagcaattcgccattactctgaagtcctacattgaatgcaatcggccccagcctgctcataggttcttgttcctgaagatcatggctatgctcaccgagctccgcagcatcaatgctcagcacacccagcggctgctgcgcatccaggacatacacccctttgctacgcccctcatgcaggagttgttcggtatcacaggtagctgagtggctgtccttgggtgacacctccgagaggtagttagacccagagccctctgagtcgccactcccgggccaagacagatggacactgccaagagccgacaatgccctgctggcctgtctccctagggaattc

SEQ ID NO: 2 - protein, 434 aa, Homo sapiens
  steroid and xenobiotic sensing nuclear receptor (SXR)
MEVRPKESWNHADFVHCEDTESVPGKPSVNADEEVGGPQICRVCGDKATGYHFNVMTCEGCKGFFRRAMKRNARLRCPFRKGACEITRKTRRQCQACRLRKCLESGMKKEMIMSDEAVEERRALIKRKKSERTGTQPLGVQGLTEEQRMMIRELMDAQMKTFDTTFSHFKNFRLPGVLSSGCELPESLQAPSREEAAKWSQVRKDLCSLKVSLQLRGEDGSVWNYKPPADSGGKEIFSLLPHMADMSTYMFKGIISFAKVISYFRDLPIEDQISLLKGAAFELCQLRFNTVFNAETGTWECGRLSYCLEDTAGGFQQLLLEPMLKFHYMLKKLQLHEEEYVLMQAISLFSPDRPGVLQHRVVDQLQEQFAITLKSYIECNRPQPAHRFLFLKIMAMLTELRSINAQHTQRLLRIQDIHPFATPLMQELFGITGS

(19) United States
(12) Patent Application Publication
(10) Pub. No.: US 2004/0053866 A1
(43) Pub. Date: Mar. 18, 2004

(54) Tumor suppressor genes and their uses

(75) Inventors: Jeff Hager, Berkeley, CA (US); Graeme Hodgson, San Francisco, CA (US); Douglas Hanahan, San Francisco, CA (US); Joe Gray, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA

(21) Appl. No.: 10/226,997

(22) Filed: Aug. 21, 2002

(51) Int. Cl. A61K 48/00; C12Q 1/68; C07H 21/04

(52) U.S. Cl. 514/44; 435/6; 536/23.2

(57) ABSTRACT

The present invention provides methods of detecting cancer cells in a patient. The methods comprise detecting SXR function in a biological sample from the patient.

### STATEMENT AS TO RIGHTS TO INVENTIONS MADE UNDER FEDERALLY SPONSORED RESEARCH AND DEVELOPMENT

This invention was made, at least in part, with government grants from the National Institutes of Health (Grant Nos. R01CA78601 and R37CA5234). Thus, the U.S. government may have certain rights in this invention.

### FIELD OF THE INVENTION

This invention relates to methods of diagnosis and treatment of cancer based on the discovery of the function of SXR as a tumor suppressor

### BACKGROUND OF THE INVENTION

Lipophilic hormones (e.g., steroids) control many aspects of human growth and development. These hormones interact with a large superfamily of intracellular receptors that function as ligand-dependent and sequence-specific transcription factors. In addition to known receptors, a number of structurally related “orphan” nuclear receptors have also been described. These receptors are known as orphan receptors because they possess domains for DNA and ligand-binding but lack identified ligands.

It well known that hormone responsiveness also requires the ability to clear ligand from the blood so that target genes are not activated in the absence of stimulus. Steroid hormones are primarily inactivated by reduction and oxidation in the liver. It is also recognized that that exogenous steroids (also referred to as xenobiotic steroids) bind steroid hormone receptors and thus modulate the expression of downstream genes. A variety of these compounds activate P-450 genes and other genes in the liver responsible for their detoxification or degradation.

It is generally thought that regulation of the detoxification pathway is distinct from classic steroid hormone receptors. In particular, hepatic orphan nuclear receptors are likely involved in the induction of genes encoding P-450 and other detoxifying enzymes. Because these enzymes are induced by high doses of xenobiotics, it has been hypothesized that these receptors involved in detoxification of xenobiotics and/or elimination of endogenous steroids are broad specificity, low-affinity receptors (Blumberg et al, Genes & Dev. 12:3195-3205 (1998)).

The human orphan nuclear receptor, termed the steroid and xenobiotic receptor (SXR), and its murine orthologue, PXR, is nuclear hormone receptor that binds to and is activated by both naturally occurring steroids and xenobiotics. It is through the binding of xenobiotics and subsequent transcriptional activation of the cytochrome p450 pathway that PXR/SXR is thought to act as Xeno-Sensor, regulating metabolism of a diverse set xenobiotics, including drugs such as rifampicin. Its function in transducing the signal of naturally occurring hormones is less well understood, but PXR/SXR can bind a multitude of steroid hormones, many of which can modulate cellular parameters such as cell proliferation and death.

Perturbation in various steroid hormone/nuclear receptor-signalling pathways has been documented in a variety of human cancers. Notably, the sex hormones, estrogen and androgen, have been shown to affect the growth of breast and prostate cancer, respectively. Both of these hormones fall into a “growth stimulatory” class in that they typically induce proliferation of cells that express their cognate receptors. Another class of ‘nuclear’ ligand receptors have anti-proliferative effects, in that they can induce apoptosis and/or initiate a differentiation program; e.g. glucocorticords and retinoic acid. Retinoids have demonstrable anti-cancer activity in multiple mouse model systems, and clinical trials are underway to determine efficacy of these molecules as chemoprevention agents.

The identification and characterization of genes in signaling pathways associated with human cancers is important in the development of new diagnostics and therapies for human cancer. The present invention addressed these and other needs.

### BRIEF SUMMARY OF THE INVENTION

The present invention provides method of detecting cancer cells in a patient. The methods comprise detecting SXR function in a biological sample from the patient. SXR function can be detected in a variety of ways. For example, SXR function can be detected by detecting the presence or absence of a functional SXR gene in the biological sample. A functional SXR gene can be conveniently detected using nucleic acid hybridization and a nucleic acid probe that specifically hybridizes to the functional SXR gene. In other embodiments, SXR function is detected by detecting the presence or absence of a functional SXR gene product (e.g., an mRNA or an SXR polypeptide) in the biological sample. SXR polypeptides can be detected using an antibody.

The invention also provides methods of inhibiting proliferation of a cell lacking SXR function. These method comprise enhancing SXR activity in the cell. A number of means of enhancing SXR activity can be used. SXR function can be enhanced by introducing into the cell a nucleic acid molecule comprising a sequence encoding an SXR polypeptide at least 80% identical to SEQ ID NO: 2. The nucleic acid molecule can be introduced into the cell using a number of known vectors, such as viral vectors, or the nucleic acid molecule can be complexed with a cationic lipid when introduced into the cell. SXR function can also be enhanced by contacting the cell with a modulator of SXR, such as an SXR ligand. Delivery of SXR polypeptides can also be used to enhance SXR function in the cell.

The invention also provides methods of inhibiting angiogenesis in a patient by enhancing SXR function in the patient. The methods of enhancing SXR function mentioned above can be used for this purpose.

Methods of promoting angiogenesis in a patient can be carried out by administering to the patient a pharmaceutical composition that inhibits SXR function in the patient.

### DEFINITIONS

The terms “SXR polynucleotide” and “SXR polypeptide” refer to nucleic acid and polypeptide polymorphic variants, alleles, and mutants that: (1) have a nucleotide sequence that has greater than about 60% nucleotide sequence identity, 65%, 70%, 75%, 80%, 85%, 90%, usually 91%, 92%, 93%, 94%, 95%, 96%, 97%, 98% or 99% or greater nucleotide sequence identity, preferably over a region of over a region of at least about 25, 50, 100, 200, 500, 1000, or more nucleotides, to SEQ ID NO: 1; (2) bind to antibodies, e.g., polyclonal antibodies, raised against an immunogen comprising SEQ ID NO: 2; (3) specifically hybridize under stringent hybridization conditions (as defined below) to SEQ ID NO: 1 (4) have an amino acid sequence that has greater than about 60% amino acid sequence identity, 65%, 70%, 75%, 80%, 85%, 90%, usually 91%, 92%, 93%, 94%, 95%, 96%, 97%, 98% or 99% or greater amino sequence identity, preferably over a region of over a region of at least about 25, 50, 100, 200, or more amino acid, to SEQ ID NO: 2. An “SXR polypeptide” and a “SXR polynucleotide,” include both naturally occurring and genetically engineered forms.

The term “lacking SXR function” refers to cells that lack normal, wild-type SXR function. Loss of function can arise from a number of causes. The particular causes are not a critical aspect of the invention. For example, loss of function can arise from loss of the SXR gene. Alternatively, loss of function can arise from mutations in the gene that lead to expression of non-functional polypeptides. Another possibility is that other genes that control expression of SXR genes are lost or altered such that expression of SXR is either lost or lacks proper control so that SXR function is effectively lost in the cell.

As used herein “SXR function”, “functional SXR polypeptide” or grammatical equivalents refer to functional SXR polypeptides that contain all of the elements required for normal function of the wild-type, full length protein as determined using a functional assay described below. A “Functional SXR gene” or grammatical equivalent refers to nucleic acids that encode functional SXR polypeptides. For example, a functional SXR polypeptide of the invention will bind an SXR ligand molecule with substantially the same affinity as the wild-type protein. Alternatively, a functional SXR polypeptide of the invention will activate transcription of steroid inducible cytochrome P-450 genes in the substantially the same manner as the wild-type protein. One of skill will recognize, however, that a functional protein need not be full length or have the wild-type sequence to provide normal function.

“Biological sample” as used herein is a sample of biological tissue or fluid that contains nucleic acids or polypeptides, and can be tested for the presence of an SXR protein, polynucleotide or transcript. Such samples include, but are not limited to, tissue isolated from primates, e.g., humans, or rodents, e.g., mice, and rats. Biological samples may also include sections of tissues such as biopsy and autopsy samples, frozen sections taken for histologic purposes, blood, plasma, serum, sputum, stool, tears, mucus, hair, skin, etc. Biological samples also include explants and primary and/or transformed cell cultures derived from patient tissues. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish. In some cases, the nucleic acids in the sample may be amplified using standard techniques such as PCR. For embodiments in which in situ hybridization techniques are used, the sample may be prepared such that individual nucleic acids remain substantially intact and typically comprises interphase nuclei prepared according to standard techniques.

The terms “identical” or percent “identity,” in the context of two or more nucleic acids or polypeptide sequences, refer to two or more sequences or subsequences that are the same or have a specified percentage of amino acid residues or nucleotides that are the same as measured using a BLAST or BLAST 2.0 sequence comparison algorithms with default parameters described below, or by manual alignment and visual inspection (see, e.g., NCBI web site or the like). This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)). [00201 Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode the same protein. Such nucleic acid variations are “silent variations,” which are one species of conservatively modified variations. Every nucleic acid sequence herein which encodes a polypeptide also describes silent variations of the nucleic acid.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor & Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that often form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed, usually by the noncovalent association of independent tertiary units. Anisotropic terms are also known as energy terms.

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. A nucleic acid of the present invention will generally contain phosphodiester bonds, although in some cases, nucleic acid analogs are included that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and peptide nucleic acid backbones and linkages.

The nucleic acids may be single stranded or double stranded, as specified, or contain portions of both double stranded or single stranded sequence. As will be appreciated by those in the art, the depiction of a single strand also defines the sequence of the complementary strand; thus the sequences described herein also provide the complement of the sequence. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, and the like.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The labels may be incorporated into the breast cancer nucleic acids, proteins and antibodies at any position. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

The term “probe” or a “nucleic acid probe”, as used herein, is defined to be a collection of one or more nucleic acid fragments whose hybridization to a sample can be detected. The probe may be unlabeled or labeled as described below so that its binding to the target or sample can be detected. Particularly in the case of arrays, either probe or target nucleic acids may be affixed to the array. Whether the array comprises “probe” or “target” nucleic acids will be evident from the context. Similarly, depending on context, either the probe, the target, or both can be labeled. The probe is produced from a source of nucleic acids from one or more particular (preselected) portions of the genome, e.g., one or more clones, an isolated whole chromosome or chromosome fragment, or a collection of polymerase chain reaction (PCR) amplification products. The probes of the present invention are produced from nucleic acids found in the regions described herein. The probe or genomic nucleic acid sample may be processed in some manner, e.g., by blocking or removal of repetitive nucleic acids or enrichment with unique nucleic acids. The word “sample” may be used herein to refer not only to detected nucleic acids, but to the detectable nucleic acids in the form in which they are applied to the target, e.g., with the blocking nucleic acids, etc. The blocking nucleic acid may also be referred to separately. What “probe” refers to specifically is clear from the context in which the word is used. The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23: 1087-1092; Kern (1997) Biotechniques 23: 120-124; U.S. Pat. No. 5,143,854). One of skill will recognize that the precise sequence of the particular probes described herein can be modified to a certain degree to produce probes that are “substantially identical” to the disclosed probes, but retain the ability to specifically bind to (i.e., hybridize specifically to) the same targets or samples as the probe from which they were derived (see discussion above). Such modifications are specifically covered by reference to the individual probes described herein.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a SXR protein).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 50° C., usually at 60°, and sometimes at 65° C. Nucleic acids that do not hybridize to each other under stringent conditions are still substantially identical if the polypeptides which they encode are substantially identical. This occurs, e.g., when a copy of a nucleic acid is created using the maximum codon degeneracy permitted by the genetic code.

“Inhibitors”, “activators”, and “modulators” of SXR polynucleotide and polypeptide sequences are used to refer to activating, inhibitory, or modulating molecules or compounds identified using in vitro and in vivo assays of SXR polynucleotide and polypeptide sequences. Inhibitors are compounds that down regulate the activity or expression of SXR proteins. “Activators” are compounds that up regulate SXR gene expression or protein activity. Such compounds can include naturally occurring and synthetic ligands, antibodies, small chemical molecules and the like. Assays for inhibitors and activators include, e.g., expressing the SXR protein in vitro, in cells, or cell membranes, applying test modulator compounds, and then determining the functional effects on activity, as described above. The phrase “detecting a cancer” refers to the ascertainment of the presence or absence of cancer in an animal. “Detecting a cancer” can also refer to obtaining indirect evidence regarding the likelihood of the presence of cancerous cells in the animal or to the likelihood or predilection to development of a cancer. Detecting a cancer can be accomplished using the methods of this invention alone, or in combination with other methods or in light of other information regarding the state of health of the animal.

A “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may be a non-tumorigenic cancer cell, such as a leukemia cell. Cancers include, but are not limited to, pancreatic cancer (e.g., pancreatic islet cancer), breast cancer, lung cancer, bronchus cancer, colorectal cancer, prostate cancer, stomach cancer, ovarian cancer, urinary bladder cancer, brain or central nervous system cancer, peripheral nervous system cancer, esophageal cancer, cervical cancer, a melanoma, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, and a chondrosarcoma.

“Tumor cell” refers to precancerous, cancerous, and normal cells in a tumor. A tumor can be either malignant or non-malignant. Thus, for example tumor cells can be associated with benign pathological cellular proliferation as well as malignant growth.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody or its functional equivalent will be most critical in specificity and affinity of binding. See Paul, Fundamental Immunology.

Antibodies exist, e.g., as intact immunoglobulins or as a number of well-characterized fragments produced by digestion with various peptidases. Thus, e.g., pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985); Coligan, Current Protocols in Immunology (1991); Harlow & Lane, Antibodies, A Laboratory Manual (1988); and Goding, Monoclonal Antibodies: Principles and Practice (2d ed. 1986)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

### DETAILED DESCRIPTION

This invention provides novel therapeutic and diagnostic methods for treatment and detection of cancer, as well as methods for screening for compositions which can be used to treat cancer. As shown below, the invention is based, at least in part, on the discovery that SXR and its orthologs, which were previously known to encode steroid hormone receptors also act as tumor suppressors whose loss facilitates progression of carcinogenesis.

The data presented here further demonstrates that SXR is involved in the control of angiogenesis. Thus, the invention also provides methods of modulating angiogenesis. For example, methods of restoring or enhancing SXR function can be used to inhibit angiogenesis to treat cancer and other conditions in which angiogenesis is deleterious. Alternatively, compounds that inhibit SXR function can be used to promote angiogenesis to treat conditions in which revascularization is desired. Pathological states for which it may be desirable to increase angiogenesis include stroke, heart disease, infertility, ulcers, wound healing, and ischemia.

Methods of Screening for Loss of Functional SXR Genes

In one aspect, SXR genes (or their expression levels) are detected in different patient samples for which either diagnosis or prognosis information is desired. For example, the presence of cancer is evaluated by a determination of the loss of functional SXR genes in the patient. Methods of evaluating the presence and/or copy number of a particular gene or to determine the presence or absence of polymorphisms in the gene are well known to those of skill in the art. For example, hybridization based assays can be used for these purposes. Alternatively, sequencing SXR proteins or genes isolated from a biological sample can be used to determine the presence of absence of polymorphisms in the gene.

Hybridization-Based Assays

Hybridization assays can be used to detect copy number or to determine the presence of polymorphisms associated with loss of SXR function. Hybridization-based assays include, but are not limited to, traditional “direct probe” methods such as Southern blots or in situ hybridization (e.g., FISH), and “comparative probe” methods such as comparative genomic hybridization (CGH). The methods can be used in a wide variety of formats including, but not limited to substrate—(e.g. membrane or glass) bound methods or array-based approaches as described below.

In a typical in situ hybridization assay, cells or tissue sections are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained.

The probes are typically labeled, e.g., with radioisotopes or fluorescent reporters. Preferred probes are sufficiently long so as to specifically hybridize with the target nucleic acid(s) under stringent conditions. The preferred size range is from about 200 bp to about 1000 bases.

In some applications it is necessary to block the hybridization capacity of repetitive sequences. Thus, in some embodiments, tRNA, human genomic DNA, or Cot-1 DNA is used to block non-specific hybridization.

In comparative genomic hybridization methods a first collection of (sample) nucleic acids (e.g. from a possible tumor) is labeled with a first label, while a second collection of (control) nucleic acids (e.g. from a healthy cell/tissue) is labeled with a second label. The ratio of hybridization of the nucleic acids is determined by the ratio of the two (first and second) labels binding to each fiber in the array. Where there are chromosomal deletions or multiplications, differences in the ratio of the signals from the two labels will be detected and the ratio will provide a measure of the copy number.

Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In Situ Hybridization Protocols, Choo, ed., Humana Press, Totowa, N.J. (1994), etc. In one particularly preferred embodiment, the hybridization protocol of Pinkel et al. (1998) Nature Genetics 20: 207-211, or of Kallioniemi (1992) Proc. Natl Acad Sci USA 89:5321-5325 (1992) is used.

A variety of nucleic acid hybridization formats are known to those skilled in the art. For example, common formats include sandwich assays and competition or displacement assays. Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

Typically, labeled signal nucleic acids are used to detect hybridization. The labels may be incorporated by any of a number of means well known to those of skill in the art. Means of attaching labels to nucleic acids include, for example nick translation, or end-labeling by kinasing of the nucleic acid and subsequent attachment (ligation) of a linker joining the sample nucleic acid to a label (e.g., a fluorophore). A wide variety of linkers for the attachment of labels to nucleic acids are also known. In addition, intercalating dyes and fluorescent nucleotides can also be used.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent labels (e.g., fluorescein, texas red, rhodamine, green fluorescent protein, and the like, see, e.g., Molecular Probes, Eugene, Oreg., USA), radiolabels (e.g., ³H ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The label may be added to the nucleic acids prior to, or after the hybridization. So called “direct labels” are detectable labels that are directly attached to or incorporated into the sample or probe nucleic acids prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

The methods of this invention are particularly well suited to array-based hybridization formats. For a description of one preferred array-based hybridization system see Pinkel et al. (1998) Nature Genetics, 20: 207-211.

Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

The DNA used to prepare the arrays of the invention are not critical. For example the arrays can include genomic DNA, e.g. overlapping clones that provide a high resolution scan of a portion of the genome containing the desired gene, or of the gene itself. Genomic nucleic acids can be obtained from, e.g., HACs, MACs, YACs, BACs, PACs, P1s, cosmids, plasmids, inter-Alu PCR products of genomic clones, restriction digests of genomic clones, cDNA clones, amplification (e.g., PCR) products, and the like.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays.

Amplification-Based Assays.

In other embodiments, amplification-based assays can be used to measure SXR gene copy number in a sample. In such amplification-based assays, the nucleic acid sequences act as a template in an amplification reaction (e.g. Polymerase Chain Reaction (PCR). In a quantitative amplification, the amount of amplification product will be proportional to the amount of template in the original sample. Comparison to appropriate (e.g. healthy tissue) controls provides a measure of the copy number.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). The known nucleic acid sequence for the genes is sufficient to enable one of skill to routinely select primers to amplify any portion of the gene.

Other suitable amplification methods include, but are not limited to ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

Detection of SXR Gene Expression

SXR gene expression level can also be assayed as a marker for cancer. In preferred embodiments, activity of the SXR gene is determined by a measure of gene transcript (e.g. mRNA), by a measure of the quantity of translated protein, or by a measure of gene product activity.

Methods of detecting and/or quantifying the gene transcript (mRNA or cDNA) using nucleic acid hybridization techniques are known to those of skill in the art (see Sambrook et al. supra). For example, one method for evaluating the presence, absence, or quantity of mRNA involves a Northern blot transfer.

The probes can be full length or less than the full length of the nucleic acid sequence encoding the protein. Shorter probes are empirically tested for specificity. Preferably nucleic acid probes are 20 bases or longer in length. (See Sambrook et al. for methods of selecting nucleic acid probe sequences for use in nucleic acid hybridization.) Visualization of the hybridized portions allows the qualitative determination of the presence or absence of mRNA.

In another preferred embodiment, a transcript (e.g., mRNA) can be measured using amplification (e.g. PCR) based methods as described above for directly assessing copy number of DNA. In a preferred embodiment, transcript level is assessed by using reverse transcription PCR (RT-PCR).

The “activity” of an SXR gene can also be detected and/or quantified by detecting or quantifying the expressed SXR polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like. The isolated proteins can also be sequence according to standard techniques to identify polymorphisms.

The SXR polypeptide is detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 377: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (polypeptide or subsequence). The capture agent is a moiety that specifically binds to the analyte. In a preferred embodiment, the capture agent is an antibody that specifically binds a polypeptide. The antibody (anti-peptide) may be produced by any of a number of means well known to those of skill in the art.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled anti-antibody. Alternatively, the labeling agent may be a third moiety, such as another antibody, that specifically binds to the antibody/polypeptide complex.

In one preferred embodiment, the labeling agent is a second human antibody bearing a label. Alternatively, the second antibody may lack a label, but it may, in turn, be bound by a labeled third antibody specific to antibodies of the species from which the second antibody is derived. The second can be modified with a detectable moiety, e.g., as biotin, to which a third labeled molecule can specifically bind, such as enzyme-labeled streptavidin.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Either polyclonal or monoclonal antibodies may be used in the immunoassays of the invention described herein. Polyclonal antibodies are preferably raised by multiple injections (e.g. subcutaneous or intramuscular injections) of substantially pure polypeptides or antigenic polypeptides into a suitable non-human mammal. The antigenicity of peptides can be determined by conventional techniques to determine the magnitude of the antibody response of an animal that has been immunized with the peptide. Generally, the peptides that are used to raise the anti-peptide antibodies should generally be those which induce production of high titers of antibody with relatively high affinity for the polypeptide.

Preferably, the antibodies produced will be monoclonal antibodies (“mAb's”). For preparation of monoclonal antibodies, immunization of a mouse or rat is preferred.

It is also possible to evaluate an mAb to determine whether it has the same specificity as a mAb of the invention without undue experimentation by determining whether the mAb being tested prevents a mAb of the invention from binding to the subject gene product isolated as described above. If the mAb being tested competes with the mAb of the invention, as shown by a decrease in binding by the mAb of the invention, then it is likely that the two monoclonal antibodies bind to the same or a closely related epitope. Still another way to determine whether a mAb has the specificity of a mAb of the invention is to preincubate the mAb of the invention with an antigen with which it is normally reactive, and determine if the mAb being tested is inhibited in its ability to bind the antigen. If the mAb being tested is inhibited then, in all likelihood, it has the same, or a closely related, epitopic specificity as the mAb of the invention.

The assays of this invention have immediate utility in detecting/predicting the likelihood of a cancer, in estimating survival from a cancer, in screening for agents that modulate the subject gene product activity, and in screening for agents that inhibit cell proliferation.

Methods of Screening for SXR Function

Assays for SXR function can be designed to detect and/or quantify any effect that is indirectly or directly under the influence of the SXR protein or nucleic acid, e.g., a functional, physical, or chemical effect. Such assays can be used to test whether a biological sample comprises a functional SXR protein, to test whether variant SXR polypeptides retain a desired SXR function, or to identify compounds that modulate SXR activity in cells.

For example the ability of SXR polypeptides to specifically bind DNA can be tested. SXR is known to heterodimerize with 9-cis retinoic acid receptor (RXR) and specifically bind direct repeats of AGGTCA or closely related sequences (see, Mangelsdorf, and Evans Cell 83:841-850 (1995)). Thus, the ability to bind these known motifs can be tested using well known techniques such as electrophoretic mobility shift assays (see, Blumberg et al., Genes & Dev. 12:3195-3205 (1998)).

Assays can also be used to detect the ability of an SXR polypeptide to activate or repress transcription of target genes, for example, steroid inducible P-450 genes. Assays for detecting gene activation are well known. For example activation of reporter genes (e.g., luciferase or GFP) linked to promoters comprising the appropriate response element can be detected (see, Blumberg et al., Genes & Dev. 12:3195-3205 (1998) and Willy, et al. Genes & Dev. 9:1033-1045 (1995)). Alternatively, chimeric receptors comprising DNA binding domains of other genes can be tested on reporter genes comprising the appropriate response element. For example, fusions of the SXR-ligand binding domain to the GAL-4 DNA binding domain can be used

Assays may include those designed to test ligand binding activity. These assays are particularly useful in identifying agents that modulate SXR activity. Virtually any agent can be tested in such an assay. Such agents include, but are not limited to natural or synthetic nucleic acids, natural or synthetic polypeptides, natural or synthetic lipids, natural or synthetic small organic molecules, and the like. In one preferred format, test agents are based on natural ligands of the SXR polypeptides, such as steroids.

Any of the assays for detecting SXR activity are amenable to high throughput screening. High throughput assays for the presence, absence, or quantification of particular nucleic acids or protein products are well known to those of skill in the art. Similarly, binding assays and reporter gene assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

As noted above, SXR modulates angiogenesis. Thus assays designed to detect the ability of SXR to modulate angiogenesis can be used e.g. the ability of isolated tissue biopsies or cells to elicit endothelial cell migration, proliferation, and tube formation in an in vitro collagen gel assay. Such assay could include those designed to assay gene expression characteristic of cells undergoing angiogenesis, and other characteristics of angiogenic cells. As noted above, inhibition of angiogenesis can be used to treat cancer. Thus, compounds that upregulate SXR activity can be screened in these assays. Alternatively, compounds that downregulate SXR activity can be screened to determine their ability to promote anglogenesis.

Other assays useful in the present invention are those designed to test other characteristics of cancer cells. These assays include cell growth on soft agar; anchorage dependence; contact inhibition and density limitation of growth; cellular proliferation; cell death (apoptosis); cellular transformation; growth factor or serum dependence; tumor specific marker levels; invasiveness into Matrigel; tumor growth and metastasis in vivo; mRNA and protein expression in cells undergoing metastasis, and other characteristics of cancer cells.

The ability of SXR polynucleotides to inhibit cell growth can also be assessed by expressing the molecules in cells lacking functional SXR genes, introducing the cells into an animal, and assessing the growth of those cells in vivo. For example, a tumor cell lacking SXR function comprising a recombinant construct of the invention can be introduced into an immunocompromised animal such as a “nude mouse” and the ability of the tumor cell to form tumors—as assessed by the number and/or size of tumors formed in the animal—is compared to the ability of a corresponding control tumor cell without the construct.

Recombinant Production of SXR Polypeptides

The present invention also provides methods, reagents, and vectors useful for expression of SXR polypeptides and nucleic acids in vitro. In vitro expression is particularly useful for production of SXR polypeptides.

Any number of well known host cells can be used for production of SXR polypeptides. Host cells may be cultured cells, cell lines, cells in vivo, and the like. Host cells may be prokaryotic cells such as bacterial cells, (e.g., E. coli), or eukaryotic cells such as yeast, insect, amphibian, or mammalian cells such as CHO, HeLa, and the like.

The particular procedure used to introduce the nucleic acids into a host cell for expression of the SXR protein is not critical to the invention. Any of the well known procedures for introducing foreign nucleotide sequences into host cells in vitro may be used. These include the use of calcium phosphate transfection, clectroporation, liposome-mediated transfection, injection and microinjection, ballistic methods, viral particles, virosomes, immunoliposomes, polycation:nucleic acid conjugates, naked DNA, artificial virions, agent-enhanced uptake of DNA, and the like.

In these embodiments of this invention, SXR nucleic acids will be inserted into vectors using standard molecular biological techniques. Vectors may be used at multiple stages of the practice of the invention, including for subcloning nucleic acids encoding components of the SXR protein as well as additional elements controlling protein expression, vector selectability, etc. Vectors may also be used to maintain or amplify the nucleic acids, for example by inserting the vector into prokaryotic or eukaryotic cells and growing the cells in culture. In addition, vectors may be used to introduce and express nucleic acids into cells for therapeutic or experimental purposes.

A variety of commercially or commonly available vectors and vector nucleic acids can be converted into a vector of the invention by cloning a nucleic acid encoding a SXR protein of the invention into the commercially or commonly available vector. A variety of common vectors suitable for this purpose are well known in the art.

In a typical embodiment, an SXR poynucleotide is placed under the control of a promoter. A nucleic acid is “operably linked” to a promoter when it is placed into a functional relationship with the promoter. For instance, a promoter or enhancer is operably linked to a coding sequence if it increases or otherwise regulates the transcription of the coding sequence. Similarly, a “recombinant expression cassette” or simply an “expression cassette” is a nucleic acid construct, generated recombinantly or synthetically, with nucleic acid elements that are capable of effecting expression of a structural gene in hosts compatible with such sequences. Expression cassettes include promoters and, optionally, introns, polyadenylation signals, and transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

An extremely wide variety of promoters are well known, and can be used in the vectors of the invention, depending on the particular application. Ordinarily, the promoter selected depends upon the cell in which the promoter is to be active. Other expression control sequences such as ribosome binding sites, transcription termination sites and the like are also optionally included. For E. coli, example control sequences include the T7, trp, or lambda promoters, a ribosome binding site and preferably a transcription termination signal. For eukaryotic cells, the control sequences typically include a promoter which optionally includes an enhancer derived from immunoglobulin genes, SV40, cytomegalovirus, a retrovirus (e.g., an LTR based promoter) etc., and a polyadenylation sequence, and may include splice donor and acceptor sequences.

For long-term, high-yield production of recombinant proteins, stable expression will often be desired. For example, cell lines which stably express a SXR protein can be prepared using expression vectors of the invention which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type. An amplification step, e.g., by administration of methyltrexate to cells transfected with a DHFR gene according to methods well known in the art, can be included.

Kits Use in Diagnostic, Research, and Therapeutic Applications

For use in diagnostic, research, and therapeutic applications disclosed here, kits are also provided by the invention. In the diagnostic and research applications such kits may include any or all of the following: assay reagents, buffers, SXR-specific nucleic acids or antibodies, hybridization probes and/or primers, and the like. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides for kits for screening for modulators of SXR. Such kits can be prepared from readily available materials and reagents. For example, such kits can comprise one or more of the following materials: an SXR polypeptide or polynucleotide, reaction tubes, and instructions for testing the desired SXR function.

A wide variety of kits and components can be prepared according to the present invention, depending upon the intended user of the kit and the particular needs of the user. Diagnosis would typically involve evaluation of a plurality of genes or products. The genes will be selected based on correlations with important parameters in disease which may be identified in historical or outcome data.

Therapeutic Methods

Administration of Modulators

The compounds that modulate SXR activity can be administered by a variety of methods including, but not limited to parenteral (e.g., intravenous, intramuscular, intradermal, intraperitoneal, and subcutaneous routes), topical, oral, local, or transdermal administration. These methods can be used for for prophylactic and/or therapeutic treatment. The pharmaceutical compositions can be administered in a variety of unit dosage forms depending upon the method of administration. For example, unit dosage forms suitable for oral administration include powder, tablets, pills, capsules and lozenges.

As noted above, modulators of the invention can be used to treat cancer and other diseases associated with pathological cellular proliferation. In these embodiments, compounds that enhance or upregulate SXR activity are used. Alternatively, compounds that inhibit SXR activity can be used to promote angiogenesis. In these embodiments, diseases in which revascularization is desired are treated. Exemplary diseases or conditions include stroke, heart disease, infertility, ulcers, wound healing, and ischemia.

The compositions for administration will commonly comprise a modulator dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs.

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

The compositions containing modulators can be administered for therapeutic or prophylactic treatments. In therapeutic applications, compositions are administered to a patient suffering from a disease (e.g., pancreatic cancer) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient.

Gene and Protein Therapy

In one application of the present invention, SXR polypeptides or recombinant nucleic acids encoding them are introduced into a patient. This form of therapy can include the use of nucleic acid vectors, or can be accomplished simply by complexing a desired nucleic acid or polypeptide with an appropriate delivery molecule. Several approaches for introducing polypeptides or nucleic acids into cells in vivo, and ex vivo have been used. The delivered nucleic acids can be used to enhance or replace SXR function (e.g., for treatment of cancer). Alternatively the delivered nucleic acids can be designed to inhibit SXR function (e.g., to promote angiogenesis). In these embodiments, the nucleic acids are used to encode antisense RNAs, iRNAs or ribozymes. Methods for designing such inhibitory nucleic acids are well known.

In protein therapy, active fragments, synthetic peptides, mimetics or other analogs of SXR are administered to the patient. The protein may be produced by recombinant expression means or, by synthetic means. Formulations are selected based on the route of administration and purpose including, but not limited to, liposomal formulations and standard pharmaceutical preparations well known to those of skill in the art.

For gene therapy, methods of delivery include lipid carrier based gene delivery, viral vector mediated gene delivery (e.g., vectors based upon infectious viruses such as retroviruses, adenoviruses, adeno-associated viruses, pox viruses, herpes viruses and many others), and delivery of naked nucleic acids. A large number of gene therapy protocols have been approved for human clinical trials, and many more have been used in animal models.

Liposome based gene delivery are known (see, e.g., U.S. Pat. Nos.5,756,353; 5,049,386, 4,946,787; and 4,897,355; PCT publications WO 91/17424, WO 91/16024; Mannino and Gould-Fogerite (1988) BioTechniques 6(7): 682-691; Rose U.S. Pat No. 5,279,833; Brigham (1991) WO 91/06309 and U.S. Pat. 5,676,954; and Felgner et al. (1987) Proc. Natl. Acad. Sci. USA 84: 7413-7414))

Adenoviral vector mediated gene delivery for treatment of cancer is describe in, e.g., Chen et al. (1994) Proc. Nat'l. Acad. Sci. USA 91: 3054-3057; Tong et al. (1996) Gynecol. Oncol. 61: 175-179; Claymanetal. (1995) Cancer Res. 5: 1-6; O'Malley et al. (1995) Cancer Res. 55: 1080-1085; Hwang et al. (1995) Am. J. Respir. Cell Mol. Biol. 13: 7-16; Haddada et al. (1995) Curr. Top. Microbiol. Immunol. 199 (Pt. 3): 297-306; Addison et al. (1995) Proc. Nat'l. Acad. Sci. USA 92: 8522-8526; Colak et al. (1995) Brain Res. 691: 76-82; Crystal (1995) Science 270: 404-410; Elshami et al. (1996) Human Gene Ther. 7: 141-148; Vincent et al. (1996) J. Neurosurg. 85: 648-654).

Replication-defective retroviral vectors harboring a therapeutic polynucleotide sequence as part of the retroviral genome have also been used, particularly with regard to simple MuLV vectors. See, e.g., Miller et al. (1990) Mol. Cell. Biol. 10:4239 (1990); Kolberg (1992) J. NIH Res. 4:43, and Cornetta et al. Hum. Gene Ther. 2:215 (1991)).

Packaged or naked nucleic acids and transduced cells (for ex vivo gene therapy) can be administered directly to a patient, preferably a human. Administration is by any of the routes normally used for introducing a molecule or cell into ultimate contact with blood or tissue cells. Packaged vector nucleic acid is administered in any suitable manner, preferably with pharmaceutically acceptable carriers. Suitable delivery methods are selected by practitioners in view of acceptable practices and regulatory requirements. It will be appreciated that the delivery methods listed above for SXR modulators may be used for transfer of nucleic acids into cells for purposes of gene therapy.

Pharmaceutically acceptable excipients are determined in part by the particular composition being administered, as well as by the particular method used to administer the composition. Accordingly, there is a wide variety of suitable formulations of pharmaceutical compositions of the present invention. Formulations suitable for parenteral administration, such as, for example, intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous administration is the preferred method of administration.

The dose administered to a patient, in the context of the present invention should be sufficient to effect a beneficial therapeutic response in the patient over time. The dose will be determined by the efficacy of the particular target nucleic acid in the vector nucleic acid and the condition of the patient, as well as the body weight or surface area of the patient to be treated. The size of the dose also will be determined by the existence, nature, and extent of any adverse side-effects that accompany the administration of a particular vector or transduced cell type in a particular patient.

For administration, vectors and transduced cells of the present invention can be administered at a rate determined by the transduced cell type, and the side-effects of the vector or cell type at various concentrations, as applied to the mass and overall health of the patient. Administration can be accomplished via single or divided doses. For a typical 70 kg patient, a dose equivalent to approximately 0.1 μg to 100 mg are administered. Transduced cells are optionally prepared for reinfusion according to established methods (see, e.g., Abrahamsen et al., J. Clin. Apheresis 6:48-53 (1991); Carter et al., J. Clin. Apheresis 4:113-117 (1988); and Aebersold et al., J. Immunol. Methods 112:1-7 (1988); see, also, Remington's Pharmaceutical Science (Gennaro et al., eds., 17th ed.)).

### EXAMPLES

The following example is offered to illustrate, but not to limit the claimed invention.

### Example 1

A transgenic mouse model of islet-cell carcinoma, RIP1-Tag2, was used to identify the association of loss of PXR function with cancer. In this model, the SV40 oncoproteins were expressed in the pancreatic islet beta cells under the control of the insulin promoter, and elicited the development of invasive carcinomas that arise through a series of distinct premalignant stages. The timing and stochastic nature in which these lesions appear strongly suggest that the transgene-derived oncoproteins while necessary, are not sufficient for tumor development; therefore, other genetic and epigenetic changes are thought to be required for tumor formation.

In a genome-wide search for loss-of heterozygosity (LOH), 2 distinct regions of loss were detected in a statistically significant manner; LOH9, confined to the distal portion of chromosome 9 and LOH16, confined to the proximal region of chromosome 16. LOH9 was detected in ˜20% of end-stage tumors whereas LOH16 ˜30%. Interestingly both of these regions are syntenic to human chromosome 3q21-25 and are only separated by ˜10 Mb. In a subsequent LOH study on distinct premalignant stages of RIP-Tag tumorigenesis, it was shown that LOH16 is preferentially lost in the transition between hyperplastic islet and angiogenic islet stages, whereas LOH9 is typically lost as angiogenic islets progress into encapsulated tumors. Based on the stage at which these losses occur it was inferred that LOH16 is involved in regulating angiogenesis and LOH 9 in down-regulation of apoptosis.

A BAC-based array CGH technique was used to further delimit both of these regions, with the eventual goal of identifying the tumor suppressor genes that reside within. In particular, using an array that contained a “complete-coverage contig” of a 3 MB minimal region of LOH16, deletions in a series of tumors and tumor cell lines were mapped and subsequently were revealed to have to a region of common overlap of only 370 kb. Using the annotated mouse genome sequence (Celera), it was determined that only 6 genes map to this region, some of which are known genes and others that appear to be novel in that they have no significant homology to annotated sequences in the Celera or Public databases. Below are the 6 genes listed in their distal to proximal order on chromosome 16.

MCG53496: Gaba-B related receptor

MCG64103

MCG65895

MCG20595

MCG20594: PXR nuclear receptor

MCG20588: Glycogen synthase Kinase 3 beta

To gain insight into which of the 6 genes may be functioning as a tumor suppressor gene, the expression of all 6 genes was evaluated using a PCR based assay, performed on 1^{st} strand cDNA derived from RNA (RT-PCR) isolated from normal, non-transgenic islets, and hyperplastic islets, angiogenic islets and tumors from RIP-Tag mice. Inactivation of tumor suppressor genes often occurs genetically by deletion and/or mutation but also epigenetically by DNA methylation, and subsequent transcriptional silencing. Given the possibility that certain mutations may result in the destabilization of the mRNA, thereby decreasing its steady-state levels, or that methylation may result in transcriptional silencing, it was reasoned that a gene whose expression is relatively high in the normal, non-transgenic islet stage followed by progressive decrease of expression during RIP-Tag tumorigenesis, indicates tumor suppressor function. Of the 6 candidate genes whose expression was screened only 1, MCG20594, encoding PXR, a steroid and xenobiotic nuclear receptor, exhibited this pattern. Expression of the PXR gene was highest in the normal, non-transgenic islets, and exhibited a decrease in expression in the hyperplastic islet stage. Notably, PXR expression was not detected in pools of angiogenic islets or tumors or in 4 independent RIP-Tag tumor cell lines. The 5 other genes were not expressed or expressed at apparently equal levels in all stages or only in tumors, in patterns deemed inconsistent with tumor suppressor gene function.

Based on the above data, that PXR is a tumor suppressor gene whose loss facilitates progression of pancreatic islet carcinogenesis in the RIP l-Tag2 mouse model. The characteristic loss of PXR during the progression from pre-angiogenic to angiogenic neoplastic lesions further implicates PXR in the control of angiogenesis, in particular acting as a suppressor or inhibitor that helps maintain the normally quiescent tissue vasculature as such.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of detecting a cancer cell in a patient, the method comprising detecting SXR function in a biological sample from the patient.

2. The method of claim 1, wherein the SXR function is detected by detecting the presence or absence of a functional SXR gene in the biological sample.

3. The method of claim 2, wherein the functional SXR gene is detected using nucleic acid hybridization and a nucleic acid probe that specifically hybridizes to the functional SXR gene.

4. The method of claim 3, wherein the probe is on an array.

5. The method of claim 3, wherein the hybridization is in situ hybridization.

6. The method of claim 3, wherein the probe is labeled.

7. The method of claim 1, wherein the SXR function is detected by detecting the presence or absence of a functional SXR gene product in the biological sample.

8. The method of claim 7, wherein the SXR gene product is an SXR polypeptide.

9. The method of claim 8, wherein the SXR polypeptide is detected using an antibody.

10. A method of inhibiting proliferation of a cell lacking SXR function, the method comprising enhancing SXR activity in the cell.

11. The method of claim 10, wherein the step of enhancing SXR function is carried out by introducing into the cell a nucleic acid molecule comprising a sequence encoding an SXR polypeptide at least 80% identical to SEQ ID NO: 2.

12. The method of claim 10, wherein the sequence encoding the SXR polypeptide is SEQ ID NO: 1.

13. The method of claim 10, wherein the SXR polypeptide is SEQ ID NO: 2.

14. The method of claim 10, wherein the nucleic acid molecule is introduced into the cell using a viral vector.

15. The method of claim 10, wherein the nucleic acid molecule is complexed with a cationic lipid when introduced into the cell.

16. The method of claim 10, wherein the step of enhancing SXR function is carried out by contacting the cell with a modulator of SXR.

17. The method of claim 16, wherein the modulator is an SXR ligand.

18. The method of claim 17, wherein the SXR ligand is a steroid.

19. The method of claim 10, wherein the step of enhancing SXR function is carried out by introducing into the cell an SXR polypeptide comprising a sequence at least 80% identical to SEQ ID NO: 2.

20. The method of claim 19, wherein the SXR polypeptide is SEQ ID NO: 2.

21. A method of inhibiting angiogenesis in a patient, the method comprising administering to the patient a pharmaceutical composition that enhances SXR function in the patient.

22. The method of claim 21, wherein the step of enhancing SXR function is carried out by administering to the patient a nucleic acid molecule comprising a sequence encoding an SXR polypeptide at least 80% identical to SEQ ID NO: 2.

23. The method of claim 22, wherein the sequence encoding the SXR polypeptide is SEQ ID NO: 1.

24. The method of claim 22, wherein the SXR polypeptide is SEQ ID NO: 2.

25. The method of claim 22, wherein the nucleic acid molecule is introduced into the cell using a viral vector.

26. The method of claim 22, wherein the nucleic acid molecule is complexed with a cationic lipid when introduced into the cell.

27. The method of claim 22, wherein the step of enhancing SXR function is carried out by contacting the cell with a modulator of SXR.

28. The method of claim 27, wherein the modulator is an SXR ligand.

29. The method of claim 28, wherein the SXR ligand is a steroid.

30. The method of claim 22, wherein the step of enhancing SXR function is carried out by introducing into the cell an SXR polypeptide comprising a sequence at least 80% identical to SEQ ID NO: 2.

31. The method of claim 30, wherein the SXR polypeptide is SEQ ID NO: 2.

32. A method of promoting angiogenesis in a patient, the method comprising administering to the patient a pharmaceutical composition that inhibits SXR function in the patient.

33. The method of claim 32, wherein the step of inhibiting SXR function is carried out by administering an inhibitor of SXR.
